(19) United States
(12) Patent Application Publication
(10) Pub. No.: US 2011/0295348 A1
(43) Pub. Date: Dec. 1, 2011

(54) SYSTEM AND METHOD FOR PROVIDING LIGHT THERAPY TO A SUBJECT

(75) Inventors: Zachary Paul, Pittsburgh, PA (US); Michael Edward Colbaugh, Level Green, PA (US)

(73) Assignee: Koninklijke Philips Electronics N.V., Eindhoven (NL)

(21) Appl. No.: 13/148,118

(22) PCT Filed: Jan. 28, 2010
(86) PCT No.: PCT/IB2010/050391

§ 371 Date: Aug. 9, 2011

Related U.S. Application Data
(60) Provisional application No. 61/152,028, filed on Feb. 12, 2009.

(51) Int. Cl. A61N 5/06

(52) U.S. Cl. 607/90

(57) ABSTRACT

A light therapy device is configured to deliver light therapy to a subject. Light therapy is delivered to the subject in the form of electromagnetic radiation that is emitted by the light therapy device onto the face of the subject in accordance with a predetermined light therapy algorithm. The light therapy device is configured to have a form factor that facilitates use by the subject. More particularly, the form factor of the light therapy device enables the light therapy device to be used discretely and comfortably while the subject is awake and/or asleep.

This patent application claims the priority benefit under 35 U.S.C. §119(e) of U.S. Provisional Application No. 61/152,028 filed on Feb. 12, 2009, the contents of which are herein incorporated by reference.

This application is related to U.S. Patent Application Ser. No. 61/141,273, entitled “SYSTEM AND METHOD FOR PROVIDING LIGHT THERAPY TO A SUBJECT,” filed Dec. 30, 2008, and U.S. patent application Ser. No. 61/141,274 entitled “SYSTEM AND METHOD FOR PROVIDING LIGHT THERAPY TO A SUBJECT,” filed Dec. 30, 2008, and U.S. patent application Ser. No. 61/141,289 entitled “SYSTEM AND METHOD FOR ADMINISTERING LIGHT THERAPY,” filed Dec. 30, 2008, and U.S. patent application Ser. No. 61/141,292 entitled “SYSTEM AND METHOD FOR ADMINISTERING LIGHT THERAPY,” filed Dec. 30, 2008, and U.S. patent application Ser. No. 61/141,295 entitled “SYSTEM AND METHOD FOR ADMINISTERING LIGHT THERAPY,” filed Dec. 30, 2008, which are hereby incorporated into this application in its entirety.

The invention relates to a device configured to provide light therapy to a subject.

The direction of radiation on a subject to impact the Circadian rhythms and/or to address light deficient disorders of the subject are known. Generally, these treatments involve shining light directly towards a patient's eyes while the patient is awake to alleviate or cure light deficient disorders including Seasonal Affective Disorder (SAD), circadian sleep disorders and circadian disruptions associated with jet-lag, and shift-work.

There are two types of light therapy devices presently available. One type of device is large in size and floor or desk mountable. These devices include light sources of fluorescent bulbs or large arrays of light emitting diodes. Although they can be moved from one position to another, they are not generally portable and require a scheduled time period of being stationary during the active part of the day. In addition, the light source is quite fragile. The second kind of light therapy device is head mountable. These devices are formed as eyeglasses or visors. While they are portable, they are not generally accepted by patients for use in public because of their odd appearance when worn on the head, and/or because of their interference with everyday activity.

One aspect of the invention relates to a light therapy device configured to provide light therapy to a subject. In one embodiment the device comprises one or more lighting modules and one or more adhesive surfaces. The one or more lighting modules are configured to at least partially surround an eye of a subject, and to emit electromagnetic radiation. The one or more adhesive surfaces are configured to hold the one or more lighting modules in place surrounding the eye of the subject.

Another aspect of the invention relates to a method of providing light therapy to a subject. In one embodiment, the method comprises holding one or more lighting modules in place to at least partially surround an eye of a subject, wherein the holding in place of the one or more lighting modules is accomplished via adhesion to the face of the subject; and emitting electromagnetic radiation from the one or more lighting modules onto the eye of the subject.

Another aspect of the invention relates to a system configured to provide light therapy to a subject. In one embodiment, the system comprises means for holding one or more lighting modules in place to at least partially surround an eye of a subject, wherein the holding in place of the one or more lighting modules is accomplished via adhesion to the face of the subject; and means for emitting electromagnetic radiation from the one or more lighting modules onto the eye of the subject.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. In one embodiment of the invention, the structural components illustrated herein are drawn to scale. It is to be expressly understood, however, that the drawings are for the purpose of illustration and description only and are not a limitation of the invention. In addition, it should be appreciated that structural features shown or described in any one embodiment herein can be used in other embodiments as well. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

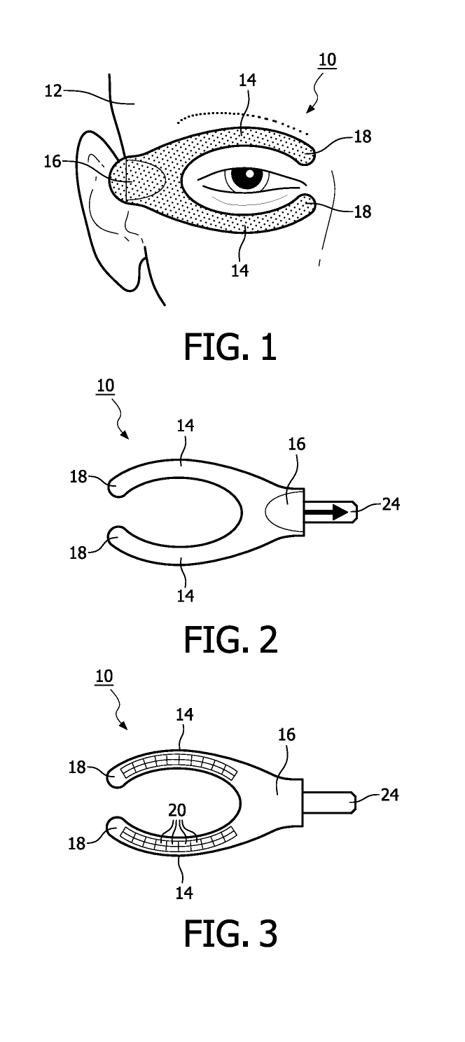

FIG. 1 illustrates a light therapy device, according to one or more embodiments of the invention.

FIG. 2 illustrates a light therapy device, according to one or more embodiments of the invention.

FIG. 3 illustrates a light therapy device, according to one or more embodiments of the invention.

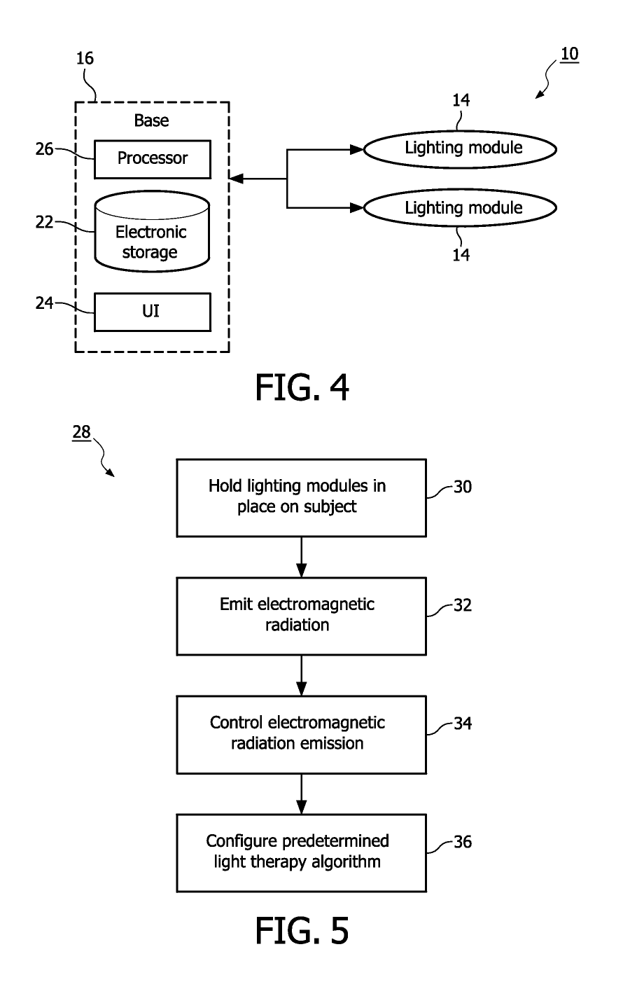

FIG. 4 illustrates a light therapy device, according to one or more embodiments of the invention.

FIG. 5 illustrates a method of providing light therapy to a subject, in accordance with one or more embodiments of the invention.

FIG. 1 illustrates a light therapy device 10, in accordance with one embodiment of the invention. Light therapy device 10 is configured to deliver light therapy to a subject 12. Light therapy is delivered to subject 12 in the form of electromagnetic radiation that is emitted by light therapy device 10 onto the face of subject 12 in accordance with a predetermined light therapy algorithm. The predetermined light therapy algorithm may dictate various aspects of the emission of the electromagnetic radiation. For example, the predetermined light therapy algorithm may dictate one or more of a timing, duration, intensity, area of incidence, emission wavelength, and/or other aspects of the emission of electromagnetic radiation by light therapy device 10. Light therapy device 10 is configured to have a form factor that facilitates use by subject 12. More particularly, the form factor of light therapy device 10 enables light therapy device 10 to be used discretely and comfortably while subject 12 is awake and/or asleep. In one embodiment, light therapy device 10 includes one or more lighting modules 14 and base 16.

In one embodiment, lighting modules 14 are formed as elongated members that extend from base 16 to distal ends 18. Lighting modules 14 may be formed from a relatively flexible material that enables subject 12 to customize the shape of lighting modules 14 between base 16 and distal ends 18. For example, subject 12 may conform the shape of lighting modules 14 to the shape of the eye of subject 12 so that lighting modules 14 sit on or just inside the orbital of the eye. In one embodiment, lighting modules 14 are formed from a relatively rigid material that does not enable customization of the shape of lighting modules 14.

As can be seen in FIGS. 1 and 2, on a front side (e.g., the side that faces outward from subject 12 during use), lighting modules 14 may be opaque. The opacity of the front side of lighting modules 14 will tend to decrease the visual obtrusiveness of light therapy device 10 to people interacting with subject 12 while subject 12 is awake, and to decrease the obtrusiveness of light therapy device 10 to people sleeping in the same room and/or bed as subject 12. In one embodiment, the front side of lighting modules 14 is a muted tone, such as a flesh-colored tone.

As can be seen in FIG. 3, a back side (e.g., the side that faces toward subject 12 during use) of lighting modules 14 are transparent, or at least translucent, to enable electromagnetic radiation generated within lighting modules 14 to be transmitted onto the face/eye of subject 12. In one embodiment, lighting modules 14 carry therein a plurality of radiation sources 20. Radiation sources 20 may comprise one or more of Organic Light Emitting Diodes (“OLEDs”), lasers (e.g., diode lasers or other laser sources), LEDs, directed ambient radiation, and/or other electromagnetic radiation sources. In one implementation, radiation sources 20 include one or more LEDs. While, the present invention is by no means limited to the use of LEDs, advantages of implementing LEDs as radiation sources 20 include their light weight, compactness, low power consumption, low voltage requirements, low heat production, reliability, ruggedness, relatively low cost, and stability. Also they can be switched on and off very quickly, reliably, and reproducibly.

Radiation sources 20 may be positioned within lighting modules 14 such that when light therapy device 10 is installed on the face of subject 12 with lighting modules 14 at least partially surrounding the eye of subject 12, electromagnetic radiation emitted from radiation sources 20 becomes incident on and/or about the eye of subject 12. In one embodiment, this includes disposing radiation sources 20 within lighting modules 14 such that electromagnetic radiation is emitted by radiation sources 20 directly toward the eye of subject 12. In one embodiment, some or all of the electromagnetic radiation emitted by radiation sources 20 is not emitted directly toward subject 12. In this embodiment, optical elements are disposed within and or formed within lighting modules 14 to direct (e.g., through reflection, refraction, diffraction, etc.) electromagnetic radiation emitted by radiation sources 20 away from the eye of the user.

In one embodiment (not shown), radiation sources 20 are not disposed along lighting modules 14 from base 16 to distal ends 18. Instead, radiation sources 20 are disposed primarily toward and/or within base 16. In this embodiment, lighting modules 14 are formed as waveguides that direct a portion of the electromagnetic radiation emitted by radiation sources 20 at or near base 16 further along lighting modules 14 toward distal ends 18 before the electromagnetic radiation is emitted from lighting modules 14 onto the eye of subject 12.

In one embodiment, base 16 forms a housing that houses various components of light therapy device 10. For example, as is discussed below, base 16 may house one or both of a processor and/or a power source. In the embodiment shown in FIGS. 1-3, base 16 is a structural tie from which lighting modules 14 extend. However, this is not intended to be limiting. In one embodiment, base 16 may be structurally separate, and may communicate electronically with lighting modules 14 via, for example, a wired tether between the base 16 and lighting modules 14.

Returning to FIG. 1, light therapy device 10 is configured to be installed on the face of subject 12 so that lighting modules 14 at least partially surround the eye of subject 12. In one embodiment, lighting modules 14 sit on or just inside of the orbital of the eye. To hold light therapy device 10 in place on the face of subject 12, light therapy device 10 includes one or more adhesive surfaces configured to removably adhere to the face of subject 12. The one or more adhesive surfaces may be formed on the back side of lighting modules 14, base 16, or lighting modules 14 and base 16. The adhesion provided by the one or more adhesive surfaces should be strong enough to secure light therapy device 10 in place during waking hours and/or during sleep. In one embodiment, the one or more adhesive surfaces are formed by applying a medical grade adhesive to one or more surfaces of lighting modules 14 and/or base 16.

In one embodiment, light therapy device 10 illustrated in FIGS. 1-3 is disposable. For example, the adhesion used to hold lighting modules 14 and/or base 16 in place on the face of subject 12 may be intended for a limited number of uses (e.g., 1), and upon reaching this number of uses, subject 12 may dispose of light therapy device 10 and apply a fresh device 10 for the next therapy session. In one embodiment, only a portion of light therapy device 10 is disposable. For example, lighting modules 14 may be removably connected with base 16 such that after a limited number of uses (e.g., 1), subject 12 may disconnect the used lighting modules 14 and connect a fresh set of lighting modules 14 to base 16 for the next light therapy session.

It will be appreciated that the number of lighting modules 14 illustrated in FIGS. 1-3, and/or their position with respect to the eye of subject 12 is not intended to be limiting. In other embodiments, light therapy device 10 may include a different number of lighting modules 14 (e.g., one). Further, lighting modules 14 may surround more or less of the eye subject 12 than the embodiment illustrated in FIGS. 1-3.

FIG. 4 is a schematic illustration of light therapy device 10, according to one embodiment of the invention. In the embodiment shown in FIG. 4, light therapy device 10 includes, in addition to lighting modules 14 and base 16, electronic storage 22, a user interface 24, and a processor 26. Electronic storage 22, user interface 24, and/or processor 26 may be housed within base 16.

In one embodiment, electronic storage 22 comprises electronic storage media that electronically stores information. The electronically storage media of electronic storage 22 may include one or both of system storage that is provided integrally (i.e., substantially non-removable) with light therapy device 10 and/or removable storage that is removably connectable to light therapy device 10 via, for example, a port (e.g., a USB port, a firewire port, etc.) or a drive (e.g., a disk drive, etc.). Electronic storage 22 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 22 may store software algorithms, information determined by processor 26, information received via user interface 24, and/or other information that enables light therapy device 10 to function properly. Electronic storage 22 may include media provided as a separate component within light therapy device 10. Electronic storage 22 may include media provided integrally with one or more other components of light therapy device 10 (e.g., processor 26).

User interface 24 is configured to provide an interface between light therapy device 10 and the subject (and/or a caregiver) through which the subject (and/or a caregiver) may provide information to and receive information from light therapy device 10. This enables data, results, and/or instructions and any other communicable items, collectively referred to as “information,” to be communicated between the subject and processor 26. Examples of interface devices suitable for inclusion in user interface 24 include a keypad, buttons, switches, a keyboard, knobs, a tab, levers, a display screen, a touch screen, speakers, a microphone, an indicator light, an audible alarm, and/or a printer. In one embodiment, user interface 24 is designed to be visibly unobtrusive (e.g., small) in keeping with the design of light therapy device 10 for use while subject 12 is awake and/or asleep. In one embodiment, user interface 24 includes one or more portions of lighting modules 14 that can be selectively illuminated to convey information to subject 12. For example, the wavelength of the electromagnetic radiation emitted by the one or more portions, the timing of the illumination (e.g., flashing), and/or other parameters of the electromagnetic radiation emitted by the selectively illuminated portions of lighting modules 14.

It is to be understood that other communication techniques, either hard-wired or wireless, are also contemplated by the present invention as user interface 24. For example, the present invention contemplates that user interface 24 may be integrated with a removable storage interface provided by electronic storage 22. In this example, information may be loaded into light therapy device 10 from removable storage (e.g., a smart card, a flash drive, a removable disk, etc.) that enables the user(s) to customize the implementation of light therapy device 10. Other exemplary input devices and techniques adapted for use with light therapy device 10 as user interface 24 include, but are not limited to, an RS-232 port, RF link, an IR link, modem (telephone, cable or other). In short, any technique for communicating information with light therapy device 10 is contemplated by the present invention as user interface 24.

Processor 26 is configured to provide information processing and/or system control capabilities in light therapy device 10. As such, processor 26 may include one or more of a digital processor, an analog processor, a digital circuit designed to process information, an analog circuit, a state machine, and/or other mechanisms for electronically processing information and/or controlling system components. In order to provide the functionality attributed to processor 26 herein, processor 26 may execute one or more modules. The one or more modules may be implemented in software; hardware; firmware; some combination of software, hardware, and/or firmware; and/or otherwise implemented. Although processor 26 is shown in FIG. 4 as a single entity, this is for illustrative purposes only. In some implementations, processor 26 may include a plurality of processing units. These processing units may be physically located within the same device (e.g., light therapy device 10), or processor 26 may represent processing functionality of a plurality of devices operating in coordination.

In one embodiment, processor 26 controls lighting modules 14 in accordance with a predetermined light therapy algorithm. The predetermined light therapy algorithm may dictate the timing (e.g., of flashing light delivered during therapy, of the commencement of therapy, etc.), duration, the intensity, location of incidence, the wavelength, and/or other aspects of the electromagnetic radiation emitted by lighting modules 14 toward the face of subject 12 on or about the eye of subject 12. In one embodiment, the predetermined light therapy algorithm is stored in electronic storage 22, and is provided to processor 26 for execution via control of lighting modules 14. In some instances, one or more aspects of the predetermined light therapy algorithm may be adjusted or customized for the subject. Adjustments and/or customizations to the predetermined light therapy algorithm may be input to light therapy device 10 via user interface 24. In one embodiment, electronic storage 22 stores a plurality of different predetermined light therapy algorithms, and the subject (and/or a caregiver) select the predetermined light therapy algorithm that is appropriate for subject 12 via user interface 24.

As was mentioned above, in one embodiment, the predetermined light therapy algorithm may dictate the timing of the administration of radiation to the subject by light therapy device 10. As such, in this embodiment, processor 26 includes a clock. The clock may be capable of monitoring elapsed time from a given event and/or of monitoring the time of day. Subject 12 (and/or a caregiver) may be enabled to correct the time of day generated by the clock of processor 26 via, for example, user interface 24. For example, the predetermined light therapy algorithm may dictate the commencement of the delivery of electromagnetic radiation to subject 12 at a predetermined time from a specific event. For instance, to shift sleeping patterns of subject 12 forward in time (e.g., wake up later), the predetermined light therapy algorithm may dictate the commencement of delivery of electromagnetic radiation 3 hours after subject 12 goes to sleep. To achieve this timing, processor 26 may control lighting modules 14 to deliver electromagnetic radiation to the face of subject 12 approximately after a trigger input that is input by subject 12 (e.g., via user interface 24) when subject 12 goes to bed. In some instances, to shift sleeping patterns of subject 12 backward in time (e.g., wake up earlier), the predetermined light therapy algorithm may dictate the delivery of electromagnetic radiation to subject 12 4.5 hours after subject 12 goes to sleep.

In one embodiment, during therapy processor 26 controls user interface 24 to provide information to subject 12 regarding one or more aspects of the predetermined light therapy algorithm. By way of non-limiting example, where user interface 24 includes one or more sections of lighting modules 14, processor 26 controls lighting modules 14 to selectively activate the radiation sources that illuminate the one or more sections in a manner that conveys information to subject 12. For instance, processor 26 may control a section toward distal end 18 of the top lighting module 14 to flash prior to the activation of lighting modules 14 to provide electromagnetic radiation to the eye of subject 12. This warning may prepare subject 12 to receive electromagnetic radiation from lighting modules 14. In some instances, processor 26 may control one or more sections of lighting modules 14 to flash as subject 12 enters changes to the predetermined light therapy algorithm. The flashing of lighting modules 14 may provide feedback to subject 12 as to whether the changes being made to the predetermined light therapy algorithm are the changes that subject 12 is intending to make.

FIG. 5 illustrates a method 28 of providing light therapy to a subject, in accordance with one embodiment of the invention. The operations of method 28 presented below are intended to be illustrative. In some embodiments, method 28 may be accomplished with one or more additional operations not described, and/or without one or more of the operations discussed. Additionally, the order in which the operations of method 28 are illustrated in FIG. 5 and described below is not intended to be limiting.

At an operation 30, one or more lighting modules are held on the face of the subject. The one or more lighting modules may be held on the face of the subject at least partially surrounding an eye of the subject. For instance, the one or more lighting modules may be adhered to the face of the subject on or just inside the orbital. In one embodiment, operation 30 is performed by one or more adhesive surfaces that is the same as or similar to the one or more adhesive surfaces formed on lighting modules 14 and/or base 16 (shown in FIGS. 1-4 and described above).

At an operation 32, electromagnetic radiation is emitted from the one or more lighting modules onto the eye of the subject. In one embodiment, operation 32 is performed by one or more lighting modules that are the same as or similar to lighting modules 14 (shown in FIGS. 1-4 and described above).

At an operation 34, the emission of electromagnetic radiation from the one or more lighting modules is controlled in accordance with a predetermined light therapy algorithm. In one embodiment, operation 34 is performed by a processor that is the same as or similar to processor 26 (shown in FIGS. 1-4 and described above).

At an operation 36, information about one or more aspects of the predetermined light therapy algorithm are conveyed to the subject. In one embodiment, operation 36 is performed by a user interface that is the same as or similar to user interface 24 (shown in FIGS. 1-4 and described above).

At an operation 38, a user is enabled to selectively configure one or more aspects of the predetermined light therapy algorithm. In one embodiment, operation 38 is performed by a user interface that is the same as or similar to user interface 24 (shown in FIGS. 1-4 and described above).

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A light therapy device configured to provide light therapy to a subject, the device comprising:
one or more lighting modules configured to at least partially surround an eye of a subject, and to emit electromagnetic radiation; and
one or more adhesive surfaces configured to hold the one or more lighting modules in place surrounding the eye of the subject.

2. The device of claim 1, further comprising a base in electronic communication with the one or more lighting modules.

3. The device of claim 2, further comprising a processor housed within the base, the processor being configured to control the emission of electromagnetic radiation by the one or more lighting modules in accordance with a predetermined light therapy algorithm.

4. The device of claim 3, further comprising a user interface configured to convey information about one or more aspects of the predetermined light therapy algorithm to the subject.

5. The device of claim 4, wherein the user interface comprises at least a portion of one of the one or more lighting modules.

6. A method of providing light therapy to a subject, the method comprising:
holding one or more lighting modules in place to at least partially surround an eye of a subject, wherein the holding in place of the one or more lighting modules is accomplished via adhesion to the face of the subject; and
emitting electromagnetic radiation from the one or more lighting modules onto the eye of the subject.

7. The method of claim 6, further comprising adhering the one or more lighting modules to the face of the subject.

8. The method of claim 6, further comprising controlling the emission of electromagnetic radiation from the one or more lighting modules in accordance with a predetermined light therapy algorithm.

9. The method of claim 8, further comprising conveying information about one or more aspects of the predetermined light therapy algorithm to the subject.

10. The method of claim 9, wherein conveying information comprises conveying information about the one or more aspects of the predetermined light therapy algorithm to the subject via at least a portion of one of the one or more lighting modules.

11. A system configured to provide light therapy to a subject, the system comprising:
means for holding one or more lighting modules in place to at least partially surround an eye of a subject, wherein the holding in place of the one or more lighting modules is accomplished via adhesion to the face of the subject; and
means for emitting electromagnetic radiation from the one or more lighting modules onto the eye of the subject.

12. The system of claim 11, further means for comprising adhering the one or more lighting modules to the face of the subject.

13. The system of claim 11, further means for comprising controlling the emission of electromagnetic radiation from the one or more lighting modules in accordance with a predetermined light therapy algorithm.

14. The system of claim 13, further means for comprising conveying information about one or more aspects of the predetermined light therapy algorithm to the subject.

15. The system of claim 14, wherein the means for conveying information comprises means for conveying information about the one or more aspects of the predetermined light therapy algorithm to the subject via at least a portion of one of the one or more lighting modules.
